(19) United States
(12) Patent Application Publication
(10) Pub. No.: US 2011/0288522 A1
(43) Pub. Date: Nov. 24, 2011

(54) APPARATUS AND METHODS FOR DETACHING AN EXPANDABLE MEMBER FROM A MEDICAL DEVICE

(75) Inventors: Dan R. Hollowell, Longmont, CO (US); Richard W. Layne, Denver, CO (US); Jason C. Morton, Golden, CO (US); Michael Moses, Boulder, CO (US); Nickolas Romcevich, Denver, CO (US); Robert M. Scribner, Boulder, CO (US)

(73) Assignee: Crosstrees Medical, Inc., Boulder, CO (US)

(21) Appl. No.: 12/972,001

(22) Filed: Dec. 17, 2010

Related U.S. Application Data
(60) Provisional application No. 61/287,830, filed on Dec. 18, 2009.

(51) Int. Cl. A61M 25/10

(52) U.S. Cl. 604/500; 604/103.01

(57) ABSTRACT

A medical implant device includes a shaft, an expandable member coupled to the distal end portion of the shaft, a sleeve and a detachment mechanism. The expandable member is configured to move between a collapsed configuration and an expanded configuration, and can be used to deliver a medicament, a therapeutic substance and/or a structural member into the body. The sleeve is coupled to the distal end portion of the shaft, and is configured to move relative to the shaft between a first position and a second position. The sleeve is configured to be disposed about the expandable member when in the first position and disposed apart from the expandable member when in the second position. The detachment mechanism is coupled to the distal end portion of the shaft, and is configured to detach the expandable member from the shaft when the sleeve is in the second position.

### CROSS REFERENCE TO RELATED APPLICATIONS

This application claims the benefit of U.S. Provisional Application Ser. No. 61/287,830, filed on Dec. 18, 2009, entitled “Apparatus and Methods for Detaching an Expandable Member from a Medical Device,” which is incorporated herein by reference in its entirety.

### BACKGROUND

The invention relates generally to medical devices, and more particularly to methods and apparatus for filling and detaching an expandable member from a medical device.

Expandable members are used in various minimally-invasive medical procedures such as, for example, procedures to repair fractures and/or other bone defects. Expandable members are used in some known procedures to deliver a medicament for augmenting bone tissue (e.g., bone cement, bone graft material or the like) into a bone structure, such as, for example, a vertebral body. The use of expandable members to deliver such medicaments can limit the flow of medicament into certain regions within and/or outside of the vertebral body. Upon completion of some known procedures, the expandable member can be left within the vertebral body. Some known insertion tools, however, do not include a mechanism for detaching the expandable member from the insertion tool upon completion of the procedure. Moreover, because the medicament is contained within the expandable member during delivery, interdigitation of the medicament within the vertebral body can be limited.

Thus, a need exists for methods and apparatus for detaching an expandable member from a medical device. A need also exists for methods and apparatus for enhancing the positioning and interdigitation of medicaments delivered via a “leave-behind” expandable member.

### SUMMARY

Medical implant devices are described herein. In some embodiments, a medical implant device includes a shaft, an expandable member coupled to the distal end portion of the shaft, a sleeve and a detachment mechanism. The expandable member is configured to move between a collapsed configuration and an expanded configuration, and can be used to deliver a medicament, a therapeutic substance and/or a structural member into the body. The sleeve is coupled to the distal end portion of the shaft, and is configured to move relative to the shaft between a first position and a second position. The sleeve is configured to be disposed about the expandable member when the sleeve is in the first position. The sleeve is configured to be disposed apart from the expandable member when the sleeve is in the second position. The detachment mechanism is coupled to the distal end portion of the shaft, and is configured to detach the expandable member from the shaft when the sleeve is in the second position.

### BRIEF DESCRIPTION OF THE DRAWINGS

FIGS. 1-3 are schematic illustrations of a medical implant device according to an embodiment, in a first configuration, a second configuration and a third configuration, respectively.

FIGS. 4 and 5 are schematic illustrations of a medical implant device according to an embodiment, in a first configuration and a second configuration, respectively.

FIG. 6 is a perspective view of a medical implant device according to an embodiment.

FIG. 7 is a perspective view of a portion of the sleeve shown in FIG. 6 identified as region Z in FIG. 6.

FIG. 8 is a perspective view of a portion of a sleeve according to an embodiment.

FIG. 9 is a side view of the medical implant device shown in FIG. 6 in a first configuration disposed within a vertebral body.

FIG. 10 is a side view of the medical implant device shown in FIG. 6 in a second configuration disposed within the vertebral body.

FIG. 11 is a side view of the medical implant device shown in FIG. 6 in a third configuration disposed within the vertebral body.

FIG. 12 is a side view of the medical implant device shown in FIG. 6 in a fourth configuration disposed within the vertebral body.

FIG. 13 is a side view of the medical implant device shown in FIG. 6 in a fifth configuration disposed within the vertebral body.

FIGS. 14 and 15 are perspective views of is a medical implant device according to an embodiment.

FIG. 16 is a perspective view of is a medical implant device having a removably coupled expandable member according to an embodiment.

FIG. 17-19 are perspective views of is a medical implant device according to an embodiment.

FIG. 20 is a perspective view of a portion of a medical implant device having an expandable member with multiple separable portions according to an embodiment.

FIGS. 21 and 22 are cross-sectional views of a portion of a medical implant device according to an embodiment in a first configuration and a second configuration, respectively.

### DETAILED DESCRIPTION

Medical implant devices are described herein. In some embodiments, a medical implant device includes a shaft, an expandable member coupled to the distal end portion of the shaft, a sleeve and a detachment mechanism. The expandable member is configured to move between a collapsed configuration and an expanded configuration, and can be used to deliver a medicament, a pasty medicament, a therapeutic substance and/or a structural member into the body. The sleeve is coupled to the distal end portion of the shaft, and is configured to move relative to the shaft between a first position and a second position. The sleeve is configured to be disposed about the expandable member when the sleeve is in the first position. The sleeve is configured to be disposed apart from the expandable member when the sleeve is in the second position. The detachment mechanism is coupled to the distal end portion of the shaft, and is configured to detach the expandable member from the shaft when the sleeve is in the second position.

In some embodiments, an apparatus includes a shaft, an expandable member coupled to a distal end portion of the shaft, and a sleeve. The expandable member is configured to move between a collapsed configuration and an expanded configuration, and can be used to deliver a medicament, a pasty medicament, a therapeutic substance and/or a structural member into the body. The sleeve is coupled to the distal end portion of the shaft, and is configured to move relative to the shaft between a first position, a second position and a third position. The sleeve is configured to be disposed about the expandable member when the sleeve is in the first position. The sleeve is configured to be disposed apart from the expandable member when the sleeve is in the second position. The sleeve is configured to sever a portion of the expandable member when the sleeve is in the third position.

In some embodiments, an apparatus includes a shaft, an expandable member coupled to a distal end portion of the shaft, and an actuator. The expandable member includes a flexible side wall, and is configured to move between a collapsed configuration and an expanded configuration. The expandable member can be used to deliver a medicament, a pasty medicament, a therapeutic substance and/or a structural member into the body. The actuator is coupled to a proximal end portion of the shaft. The actuator is operably coupled to at least a portion of the flexible side wall, for example, by a cord, thread, filament or the like. Accordingly movement of the actuator causes the portion of the side wall to separate.

In some embodiments, a method includes conveying a medicament, a pasty medicament, a therapeutic substance and/or structural substance into an expandable member disposed within the body via a shaft coupled to the expandable member. A portion of the expandable member is then opened, thereby defining an opening within the expandable member. In some embodiments, the method optionally includes moving the expandable member within the body after the portion of the expandable member is opened. The expandable member is then detached from the shaft, and the shaft is removed from the body.

The term “expandable member” as used herein includes a component of a medical device that is configured to be changed or moved from a collapsed configuration to an expanded configuration in which the expandable member is larger than in the collapsed configuration. In some variations, the expandable member is configured to be expanded, for example, by introducing a medium such as liquid, slurry, paste and/or gas into the interior of the expandable member. The expandable member can be, for example, a balloon configured to expand from a collapsed configuration to an expanded configuration. Such balloons can be used, for example, to deliver the medium into a region within the body. In some embodiments, the medium can be a bone cement.

As used in this specification, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the end of the medical device to last enter the patient's body would be the proximal end of the medical device.

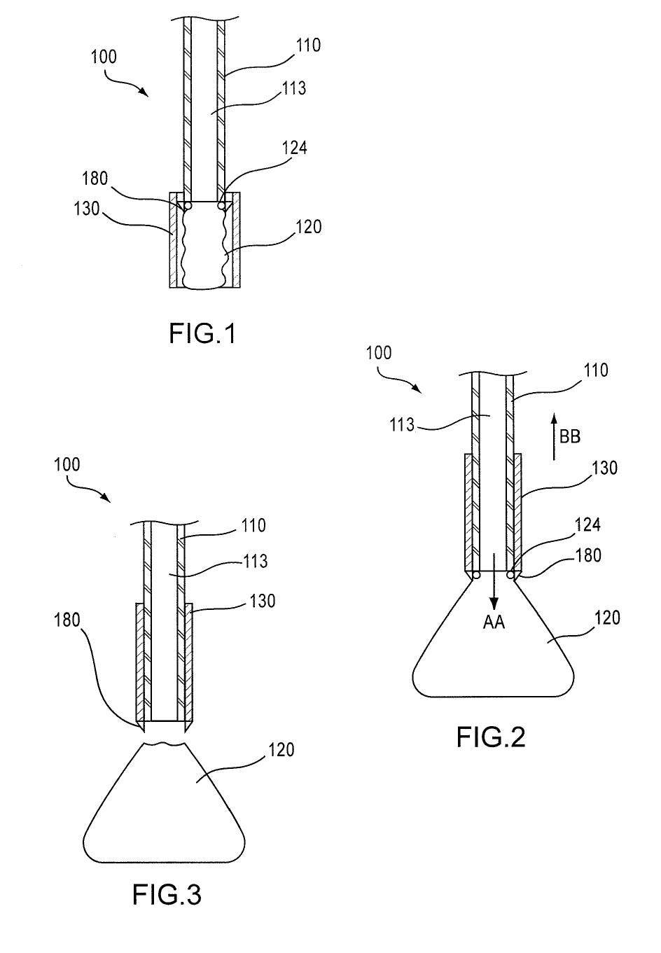

FIGS. 1-3 are schematic illustrations of a portion of a medical implant device 100 according to an embodiment of the invention, in a first configuration, a second configuration and third configuration, respectively. As described herein, in some embodiments, the medical implant device 100 can be used to deliver a bone cement into a portion of a bone (e.g., a long bone, a vertebral body or the like) to repair fractures and/or other bone defects. The bone cement can include, for example, any material configured to augment a bone structure including compositions containing polymethylmethacrylate (PMMA), barium sulfate, hydroxyapatite, monocalcium phosphate, calcium carbonate, bone chips or the like. Although described herein is being configured to deliver bone cement, in other embodiments, the medical implant device 100 can be used to deliver any suitable medicament, a pasty medicament, a therapeutic substance and/or a structural component into a body (not shown in FIGS. 1-3). In some embodiments, a medicament can include a mixture of a liquid and at least one of a medicinal powdered substance or a medicinal granular substance.

The medical implant device 100 includes a shaft 110, an expandable member 120, a sleeve 130 and a detachment mechanism 180. The expandable member 120 is coupled to the distal end portion of the shaft by a weld joint, an interference fit, an elastic coupling member, a chemical bond, or the like, as shown by interface 124. As described in more detail below, the shaft 110 defines a lumen 113 through which the bone cement can be conveyed from a region outside of the body to the interior of the expandable member 120, as shown by the arrow AA in FIG. 2. In some embodiments, the interface 124 can be a substantially hermetic and/or fluid-tight seal.

The expandable member 120 is configured to move between a collapsed configuration (see e.g., FIG. 1) and an expanded configuration (see e.g., FIGS. 2 and 3) when the bone cement is conveyed into the interior of the expandable member 120. The expandable member 120 can be any suitable expandable member, such as, for example, a non-compliant expandable member constructed from a flexible material. The flexible material can be constructed from a porous and/or woven material such that the bone cement can be conveyed from the interior of the expandable member 120 to a region outside of the expandable member. In this manner, the expandable member 120 can be constructed to enhance the interdigitation of the bone cement within the body. In some embodiments, a portion of the expandable member 120 can be separated and/or opened to enhance the interdigitation of the bone cement within the body.

The sleeve 130 is movably coupled to the distal end portion of the shaft 110, as shown by the arrow BB in FIG. 2. When the medical implant device 100 is in the first configuration (see FIG. 1), a portion of the sleeve 130 is disposed distally from the distal-most portion of the shaft 110. Thus, when the medical implant device 100 is in the first configuration (see FIG. 1), the expandable member 120 is disposed within a portion of the sleeve 130. In this manner, the sleeve 130 can protect the expandable member 120 when the distal end portion of the shaft 110 is inserted into the body.

When the medical implant device 100 is in the second configuration (see FIG. 2), the sleeve 130 is retracted (i.e., moved proximally relative to the shaft) such that the sleeve 130 is disposed apart from the expandable member 120. Similarly stated, when the medical implant device 100 is in the second configuration (see FIG. 2), the sleeve 130 is retracted such that the expandable member 120 is exposed and/or unconstrained. The sleeve 130 can be moved relative to the shaft 110 by any suitable mechanism, such as for example, by a handle (not shown in FIGS. 1-3) coupled to the proximal portion of the medical implant device 100, a electronic actuator, a magnetic actuator or the like.

When the medical implant device 100 is in the second configuration, bone cement is conveyed into the expandable member 120 through the shaft 110. Thus, the expandable member is moved from the collapsed configuration to the expanded configuration when the medical implant device 100 is in the second configuration.

After the medical implant device 100 is in the second configuration, the detachment mechanism 180 can be actuated to detach the expandable member 120 from the shaft 110, as shown in FIG. 3. In this manner, the shaft 110 can be removed from the body, leaving the expandable member 120 within the body. The detachment mechanism 180 can be any suitable mechanism for detaching the expandable member 120 from the shaft 110. The detachment mechanism 180 can include, for example, a sharp or serrated edge configured to sever a portion of the expandable member 120, a heat-activated mechanism for severing a portion of the shaft 110 and/or the expandable member 120, device configured to release a connector from the shaft 110 and/or the expandable member 120, or the like. The detachment mechanism 180 can be actuated in any suitable manner, such as for example, by a handle (not shown in FIGS. 1-3) coupled to the proximal portion of the medical implant device 100, a electronic actuator, a magnetic actuator or the like. In some embodiments, the detachment mechanism 180 can be actuated by the sleeve 130. Although the detachment mechanism 180 is shown as being separate and/or distinct from the sleeve 130, in other embodiments, the detachment mechanism 180 and the sleeve 130 can be monolithically constructed. Similarly stated, in some embodiments, a portion of the sleeve can function as a detachment mechanism.

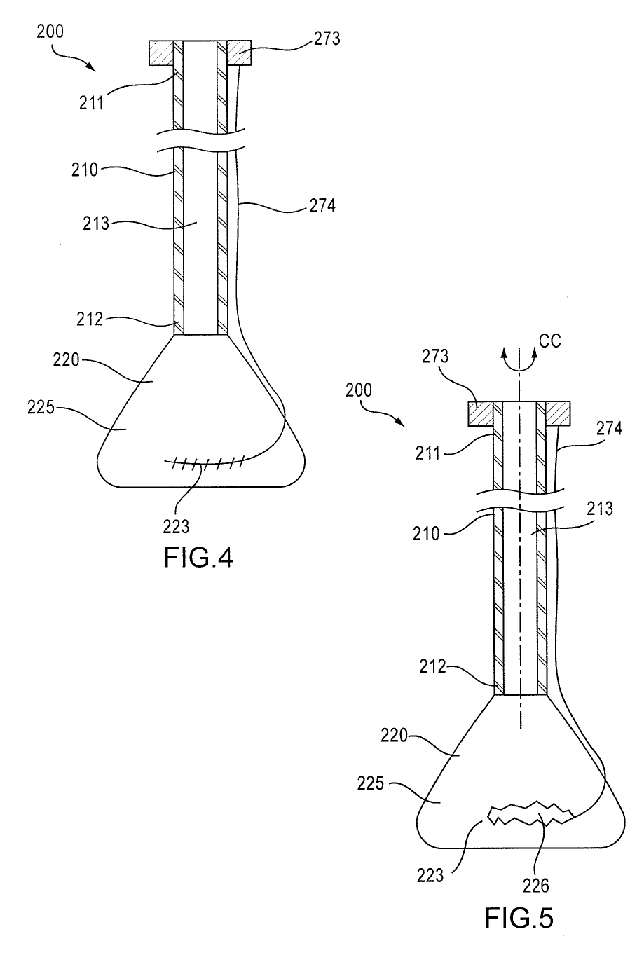

Although the detachment mechanism 180 is shown and described above as detaching the expandable member 120 from the shaft 110, in other embodiments, a medical implant device can include a device configured to separate a portion of a side wall of an expandable member. In this manner, a material contained within the expandable member can be selectively released via the separated portion of the side wall. For example, FIGS. 4 and 5 are schematic illustrations of a portion of a medical implant device 200 according to an embodiment of the invention, in a first configuration and a second configuration, respectively. As described herein, in some embodiments, the medical implant device 100 can be used to deliver a bone cement into a portion of a bone (e.g., a long bone, a vertebral body or the like) to repair fractures and/or other bone defects.

The medical implant device 200 includes a shaft 210, an expandable member 220 and an actuator 273. The shaft 210 has a proximal end portion 211 and a distal end portion 212 and defines a lumen 213. The expandable member 220 is coupled to the distal end portion 212 of the shaft 210. As described herein, a therapeutic substance can be conveyed from a region outside of the body to the interior of the expandable member 220 via the lumen 213.

The expandable member 220 is configured to move between a collapsed configuration (not shown in FIGS. 4 and 5) and an expanded configuration (see e.g., FIGS. 4 and 5) when the bone cement is conveyed into the interior of the expandable member 220. The expandable member 220 includes a side wall 225 constructed from a flexible material. The flexible material can include a porous and/or woven material such that the bone cement can be conveyed from the interior of the expandable member 220 to a region outside of the expandable member 220 through the side wall 225. For example, in some embodiments, the expandable member 220, and any of the expandable members described herein, can have porous structure of the type shown and described in U.S. Patent Publication No. 2007/0129670, entitled “Extractable Filler for Inserting Medicine into Vertebral Body,” which is incorporated herein by reference in its entirety. In other embodiments, the flexible material can be substantially impermeable. In such embodiments, the expandable member 220 can define an interior region that is substantially fluidically isolated from a region outside of the expandable member 220. Similarly stated, in such embodiments, the expandable member 220 can include a hermetic and/or fluid-tight wall.

Additionally, the side wall 225 includes a separable portion 223 configured to selectively separate (or open) when actuated. Said another way, the separable portion defines an opening 226 that is selectably releasable. In some embodiments the separable portion 223 can include perforations that can be irreversibly torn when actuated. In other embodiments, the separable portion 223 can include two edges of the side wall 225 that are reversibly fastened together, thereby defining a selectably releasable opening 226. In yet other embodiments, the separable portion 223 can include a dissolvable and/or degradable material that can define an opening in the side wall 225 when actuated. In this manner, the expandable member 220 can maintain a high pressure therein (e.g., when the separable portion 223 is closed) to enhance bone fixation, bone compaction or the like and can enhance the interdigitation of the bone cement within the body (e.g., when the separable portion 223 is opened). Similarly stated, when the separable portion 223 is closed, the separable portion 223 is substantially leakproof, thereby limiting and/or preventing flow the medicament contained within the expandable through the opening 226. Conversely, when the separable portion 223 is opened, the medicament can pass through the opening 226.

The actuator 273 is movably coupled to the proximal end portion 211 of the shaft 210. The actuator 273 is operably coupled to the separable portion 223 of the side wall 225. In particular, the actuator 273 is coupled to the separable portion 223 by a filament or thread 274. In some embodiments, the thread 274 can form and or define a portion of the separable portion 223 that maintains the opening 226 in a closed position. When the medical implant device 200 is in the first configuration (see FIG. 4), bone cement is conveyed into the expandable member 220 through the shaft 210. Additionally, the separable portion 223 of the expandable member 220 is closed, intact and/or unseparated. Similarly stated, when the medical implant device 200 is in the first configuration, the side wall 225 of the expandable member 220 substantially surrounds and/or encloses the interior region defined by the expandable member 220. Thus, in some embodiments (i.e., those in which the side wall of the expandable member is permeable and/or porous), when the medical implant device 200 is in the first configuration, the bone cement contained within the expandable member 220 can be conveyed from the internal region defined by the expandable member 220 to a region outside of the expandable member 220 through the porous side wall 225. In other embodiments, however, (i.e., those in which the side wall of the expandable member is substantially hermetic and/or fluid-tight), when the medical implant device 200 is in the first configuration, the bone cement contained within the expandable member 220 is maintained (i.e., prevented from being conveyed from) the expandable member.

The medical implant device 200 can be moved from the first configuration (FIG. 4) to the second configuration (FIG. 5) by moving the actuator 273 relative to the shaft 210 and/or the expandable member 220, as shown by the arrow CC in FIG. 5. Although the actuator 273 is shown as being rotated about a longitudinal axis of the shaft 210, in other embodiments, the actuator 273 can be moved in any direction and/or in any manner relative to the shaft 210 and/or the expandable member 220. For example, in some embodiments, the actuator 273 can be translated along the longitudinal axis of the shaft 210.

When the actuator 273 is moved, the separable portion 223 of the side wall 225 separates. Similarly stated, when the actuator 273 is moved, the separable portion 223 of the side wall 225 is opened. Said another way, movement of the actuator 273 defines an opening 226 in the side wall 225. As described above, the separable portion 223 of the side wall 225 can be separated and/or opened in any suitable manner (e.g., by tearing a portion of the side wall 225, by removing a stitch and/or thread 274, by untying a knot, by dissolution of a portion of the side wall or the like). Accordingly, when the medical implant device 200 is in the second configuration, the bone cement contained within the expandable member 220 can be conveyed from the internal region defined by the expandable member 220 to a region outside of the expandable member 220 through the opening 226.

Although the medical implant device 200 is shown and described as including a single filament or thread 274, in other embodiments, a medical implant device can include any number of threads 274 configured to selectively open and/or release a separable portion of an expandable member. Any of the medical implant devices described herein can include one or more threads of the type shown and described in U.S. Patent Publication No. 2007/0129670, entitled “Extractable Filler for Inserting Medicine into Vertebral Body,” which is incorporated herein by reference in its entirety. For example, in some embodiments, the medical implant device 200 can include two threads that are coupled together to form a knot to maintain the opening 226 in a closed position, as shown in U.S. Patent Publication No. 2007/0129670.

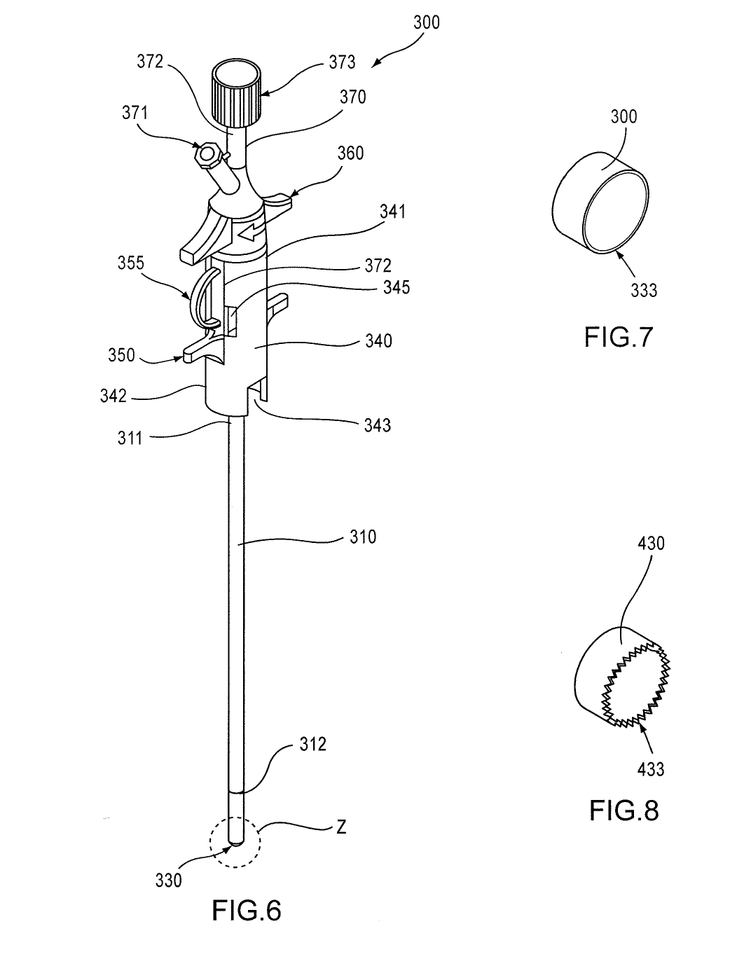

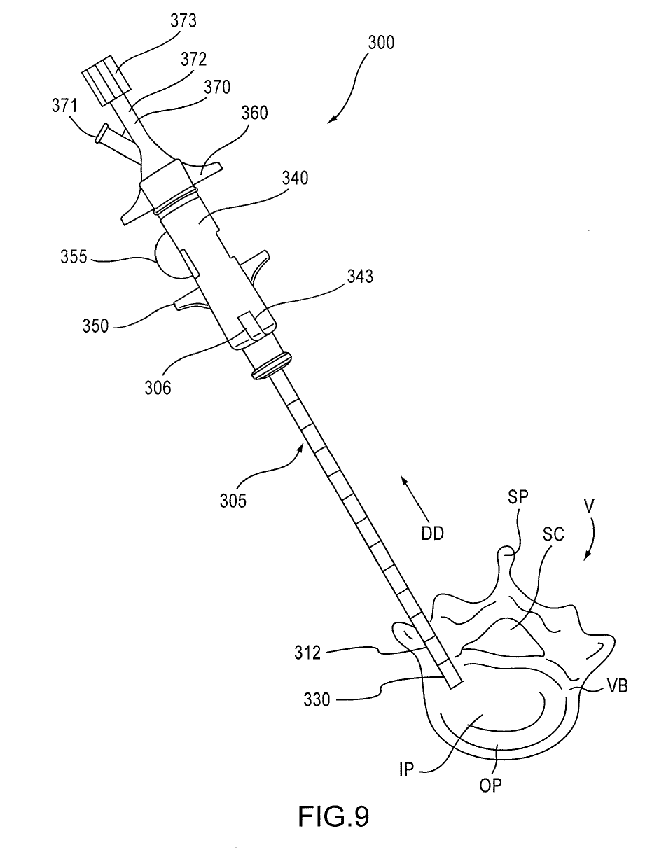

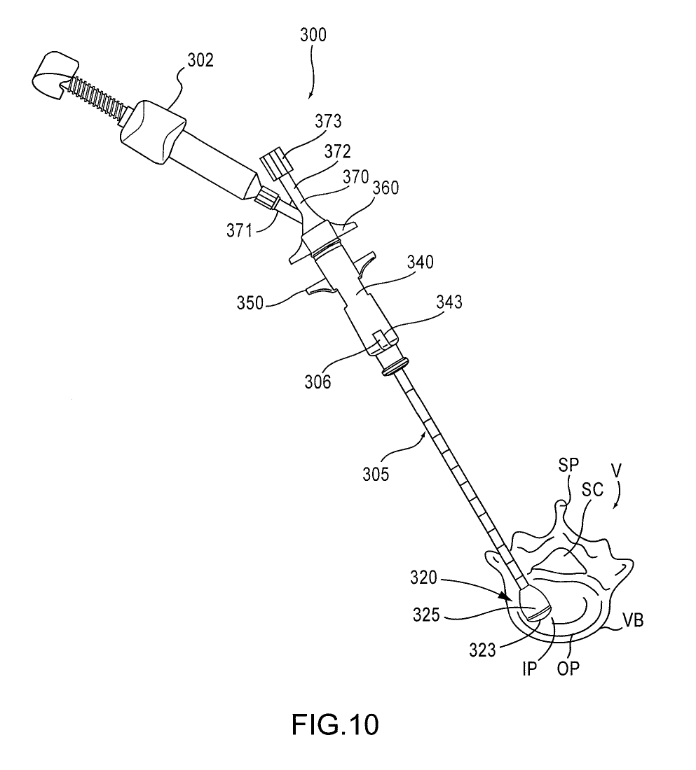

FIGS. 6-8 show a medical implant device 300 according to an embodiment, and FIGS. 9-13 show the medical implant device 300 being used to dispose an implant into a vertebra V according to an embodiment. The medical implant device 300 includes a housing 340, a shaft 310, an expandable member 320 (see e.g., FIG. 10) and a sleeve 330. The housing 340 includes a proximal end portion 341 and a distal end portion 342. The distal end portion 342 of the housing 340 defines an opening 343 configured to receive a portion of a protrusion 306 of a cannula 305 (see FIGS. 9-11). In some embodiments, the opening 343 can matingly and/or lockingly receive the protrusion 306 of the cannula 305. In this manner movement of the housing 340 relative to the cannula 305 (e.g., movement in the proximal direction) can be limited when the housing 340 is coupled the cannula 305.

The housing 340 defines at least one longitudinal opening 344 within which a sleeve actuator 350 can be moved along the longitudinal axis of the housing 340, as described in more detail below. The housing 340 further defines a circumferential opening 345 joined with the longitudinal opening 344. The circumferential opening 345 is configured to receive at least a portion of the sleeve actuator 350 when the sleeve actuator 350 is rotated about the longitudinal axis of the housing 340. In some embodiments the housing 340 can define two longitudinal openings 344 being substantially 180 degrees apart. In other embodiments, the housing 340 can define two longitudinal openings 344 spaced apart by any angle. In other embodiments, the circumferential opening 345 can be helical, thereby allowing the sleeve actuator 350 to both rotate about and translate relative to the shaft 310 when the portion of the sleeve actuator 350 travels within the circumferential opening 345.

A lock member 355 is removably disposed within the longitudinal opening 344. The lock member 355 is configured to contact and/or engage at least a portion of the sleeve actuator 350 to limit movement of the sleeve actuator within the longitudinal opening 344. In this manner, the lock member 355 can prevent inadvertent movement of the sleeve actuator 350 and/or the sleeve 330.

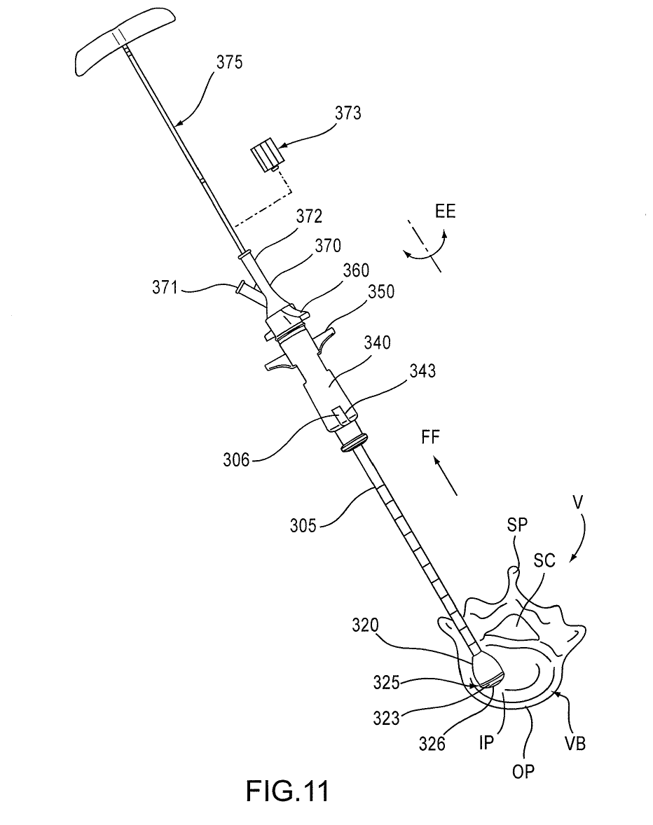

A shaft actuator 360 is movably coupled to the proximal end portion 341 of the housing 340. As described in more detail herein, rotation of the shaft actuator 360, as shown by the arrow EE in FIG. 11, results in longitudinal movement of the shaft 310 relative to the housing 340 and/or cannula 305, as shown by the arrow FF in FIG. 11.

A fill interface assembly 370 is coupled to the proximal end portion 341 of the housing 340. The fill interface assembly 370 includes a fill port 371, a stylet access port 372 and a cap 373. The fill port 371 is configured to be coupled to a pressurized source of medicament, such as, for example, the bone cement injector 302 shown in FIG. 10. The fill interface assembly 370 defines an opening therein such that source of medicament can be fluidically coupled to the shaft 310. In this manner, the medicament, therapeutic material and/or structural material to be implanted can be conveyed from the source into the shaft 310 via the fill port 371. The cap 373 is coupled to and forms a substantially fluid-tight seal with the stylet access port 372. As shown in FIG. 11, the cap 373 can be removed to allow a stylet 375 to be disposed within the shaft 310.

The shaft 310 has a proximal end portion 311 and a distal end portion 312 and defines a lumen (not shown). The proximal end portion 311 of the shaft 310 is movably coupled to the housing 340 and to the shaft actuator 360. Rotation of the shaft actuator 360, as shown by the arrow EE in FIG. 11, results in longitudinal movement of the shaft 310 relative to the housing 340 and/or cannula 305, as shown by the arrow FF in FIG. 11. In this manner, the position of the distal end portion 312 of the shaft 310 and/or the expandable member 320 within the body can be adjusted. In some embodiments the housing 340 and/or the shaft actuator 360 can include a ratchet mechanism, one or more detents and/or a threaded connection to facilitate repeatable, controlled movement of the shaft 310 relative to the housing 340 and/or the cannula 305. In some embodiments, the housing 340 and/or the shaft actuator 360 can include a dial indicator or any other suitable mechanism to indicate and/or provide feedback associated with the distance through which the shaft 310 has moved relative to the housing 340 and/or the cannula 305.

The distal end portion 312 of the shaft 310 is coupled to the expandable member 320. The expandable member 320 is configured to move between a collapsed configuration (e.g., when the expandable member 320 is disposed within the sleeve 330, see e.g. FIG. 6) and an expanded configuration (see e.g., FIGS. 10-12) when the bone cement is conveyed into the interior of the expandable member 320. The expandable member 320 includes a side wall 325 constructed from a flexible material. In some embodiments, the flexible material can include a porous and/or woven material such that the bone cement can be conveyed from the interior of the expandable member 320 to a region outside of the expandable member 320 through the side wall 325. Additionally, the side wall 325 includes a separable portion 323 configured to selectively separate (or open) when actuated. In some embodiments the separable portion 323 can include perforations that can be irreversibly torn when actuated. In other embodiments, the separable portion 323 can include two edges of the side wall 325 that are reversibly fastened together, by a stitch, a removable staple, an adhesive or the like. In yet other embodiments, the separable portion 323 can include a dissolvable and/or degradable material that can define an opening in the side wall 325 when actuated.

The sleeve 330 is movably coupled to the distal end portion of the shaft 310. More particularly, as described below, the sleeve 330 can be moved along the longitudinal axis of the shaft 310 between a first (or insertion) position, a second (or retracted) position and a third (or severing) position. The sleeve 340 is operably coupled to and moved by the sleeve actuator 350. More particularly, as described above, movement of the sleeve actuator 350 relative to the housing 340 (i.e., within the longitudinal opening 344 of the housing 340) causes movement of the sleeve 330 relative to the distal end portion 312 of the shaft. The sleeve 340 can be operably coupled to the sleeve actuator 350 by any suitable mechanism (not shown in FIG. 6). For example, in some embodiments, the sleeve 340 can be coupled to the sleeve actuator 350 by a rigid member, a flexible member (e.g., a filament, cord, or the like), and/or an elastic member.

When the sleeve 330 is in the first position (see e.g., FIG. 6), the sleeve 330 is disposed about the expandable member 320. In this manner, the sleeve 330 can protect and/or shield the expandable member 320 during insertion of the distal end portion 312 of the shaft 310 into the body. The sleeve 340 can be moved into the second position by first removing the lock member 355 from the housing 340. The sleeve actuator 340 is then moved proximally within the longitudinal opening 344 of the housing 340. In this manner, the sleeve is moved proximally along the longitudinal axis of the shaft 310, as shown by the arrow DD in FIG. 9.

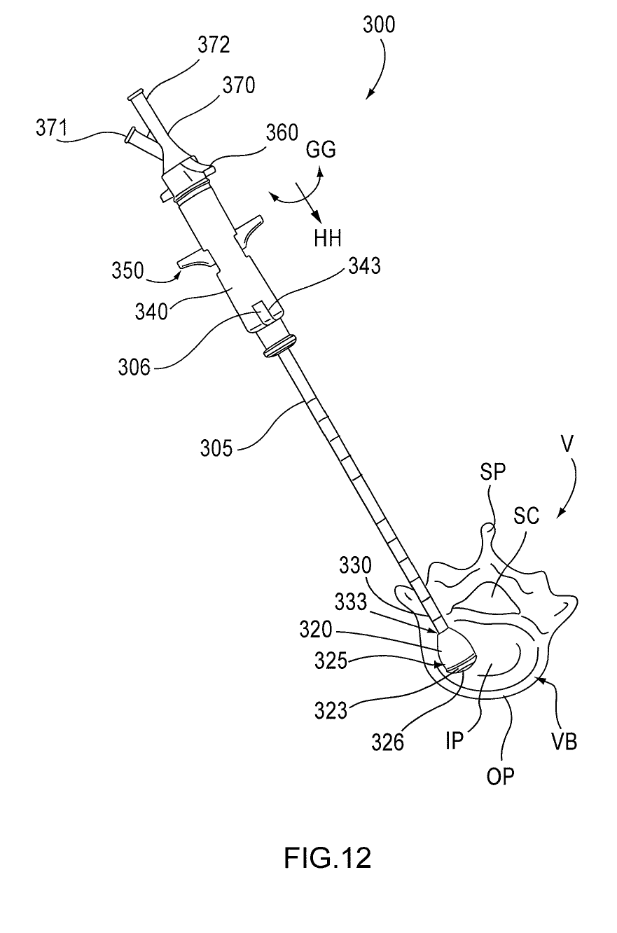

When the sleeve 330 is in the second position (see e.g., FIG. 10), the sleeve 330 is disposed apart from the expandable member 320. Similarly stated, when the sleeve 330 is in the second position, the expandable member 320 is exposed, and can therefore be moved from its collapsed configuration to its expanded configuration. The sleeve 340 can be moved into the second position by moving the sleeve actuator 340 distally within the longitudinal opening 344 of the housing 340, as shown by the arrow HH in FIG. 12.

When the sleeve 330 is in the third position (see e.g., FIG. 12), a portion of the sleeve 330 is in contact with the expandable member 320. More particularly, when the sleeve 330 is in the third position, a distal edge 333 (see e.g., FIG. 7) of the sleeve 330 is in contact with the expandable member 320. Thus, when the sleeve 330 is in the third position, the sleeve 330 can be used to detach the expandable member 320 from the shaft 310. More particularly, the sleeve 330 can be reciprocated and/or rotated relative to the shaft 310 and/or the expandable member 320 by reciprocating and/or rotating the sleeve actuator 340 within the circumferential opening 345 (as shown by the arrow GG in FIG. 12) such that the sharp or serrated distal edge 333 can sever, cut and/or tear a portion of the expandable member 320.

Although, the distal edge 333 of the sleeve 330 is shown as being sharpened to facilitate severing the expandable member 320, in other embodiments, the distal edge 333 of the sleeve 340 can have any suitable configuration. For example, FIG. 8 shows a portion of a sleeve 430 that includes a serrated or saw-tooth distal edge 433.

In some embodiments, the medical implant device 300 can be used to dispose a medical implant into a vertebra V according to an embodiment. As shown in FIGS. 9-13, the vertebra V includes a spinous process SP, a spinal cord canal SC and a vertebral body VB. The vertebral body VB includes an outer portion OP formed from a cortical bone surrounding an inner portion IP formed from cancellous bone. Although the medical implant device 300 is shown and described as being used to dispose the expandable member 320 within the vertebral body VB, in other embodiments, the medical implant device 300 can be used to dispose a medical implant within any suitable portion of the body.

In use, the medical implant device 300 is inserted into the body via the access cannula 305. More particularly, the distal end portion 312 of the shaft 310 is inserted through the outer portion OP of the vertebral body VB and into the inner portion IP. The housing 340 is coupled to the cannula 305 via the protrusion 306 and the opening 343, as described above. In this manner, the likelihood of undesired movement of distal end portion 312 of the shaft 310 within the vertebral body VB can be limited.

After the distal end portion 312 of the shaft 310 is within the vertebral body VB, the sleeve 330 is moved from its first position to its second position as shown and described above, and as shown by the arrow DD in FIG. 9. In this manner, the expandable member is exposed after being placed within the inner portion IP of the vertebral body VB. As shown in FIG. 10, a bone cement injector 302 is then coupled to the fill port 371 thereby placing the source of pressurized medicament 302 in fluid communication with the expandable member 320 via the shaft 310. The bone cement injector 302 can be coupled to the fill port 371 by any suitable mechanism, such as, for example, a Luer fitting. Although the source of pressurized medicament is shown as being a manual bone cement injection device 302, in other embodiments, the source of pressurized medicament can be any suitable pressurized source of medicament, therapeutic material and/or structural material.

Bone cement is then conveyed into the expandable member 320, thereby moving the expandable member 320 from its collapsed configuration to its expanded configuration. In some embodiments, movement of the expandable member 320 from its collapsed configuration to its expanded configuration can cause displacement and/or movement of portions of the vertebral body (e.g., compression of the cancellous bone, movement of an end plate or the like). In this manner, the expandable member 320 can be used to repair bone defects, fractures and the like.

As shown in FIG. 11, the cap 373 is removed from the fill interface assembly 370. The cap 373 is operably coupled to the separable portion 323 of the side wall 325 of the expandable member 320 by a wire, cord, filament or the like. In this manner, removal of the cap 373 causes the separable portion 323 to separate (or open). Similarly stated, removal of the cap 373 defines an opening 326 in the separable portion 323 of the expandable member 320. In this manner, the bone cement contained within the expandable member 320 can be conveyed from the internal region defined by the expandable member 320 to a region outside of the expandable member 320 to enhance the interdigitation of the bone cement within the vertebral body VB.

In some embodiments, additional bone cement is conveyed into the expandable member 320 after the opening 326 is defined therein. More particularly, as shown in FIG. 11, a stylet 375 can be disposed within the shaft 310 via the stylet access port 372. The stylet 375 can compress and/or move the bone cement within the shaft 310 into the expandable member 320. The addition of bone cement after the expandable member is opened can further enhance interdigitation of the bone cement within the vertebral body VB.

In some embodiments, the expandable member 320 can be moved within the vertebral body VB after the opening 326 is defined in the expandable member 320. More particularly, as described above, rotation of the shaft actuator 360, as shown by the arrow EE in FIG. 11, results in longitudinal movement of the shaft 310 relative to the housing 340 and/or cannula 305, as shown by the arrow FF in FIG. 11. In this manner, the position of the distal end portion 312 of the shaft 310 and/or the expandable member 320 within the body can be adjusted. In some embodiments, the expandable member 320 can be moved proximally to further enhance interdigitation of the bone cement within the vertebral body VB.

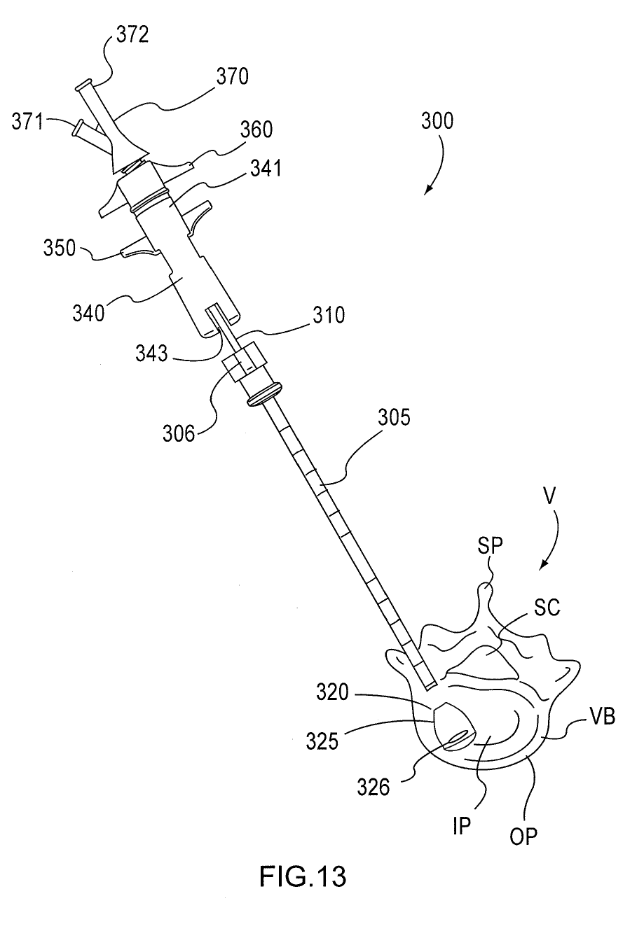

The expandable member 320 is then detached from the shaft 310 via the sleeve 330. As described above, the sleeve 330 can be reciprocated and/or rotated relative to the shaft 310 and/or the expandable member 320 by reciprocating and/or rotating the sleeve actuator 340 within the circumferential opening 345 such that the sharp distal edge 333 can sever, cut and/or tear a portion of the expandable member 320. The shaft 310 can then be removed from the vertebral body VB, as shown in FIG. 13.

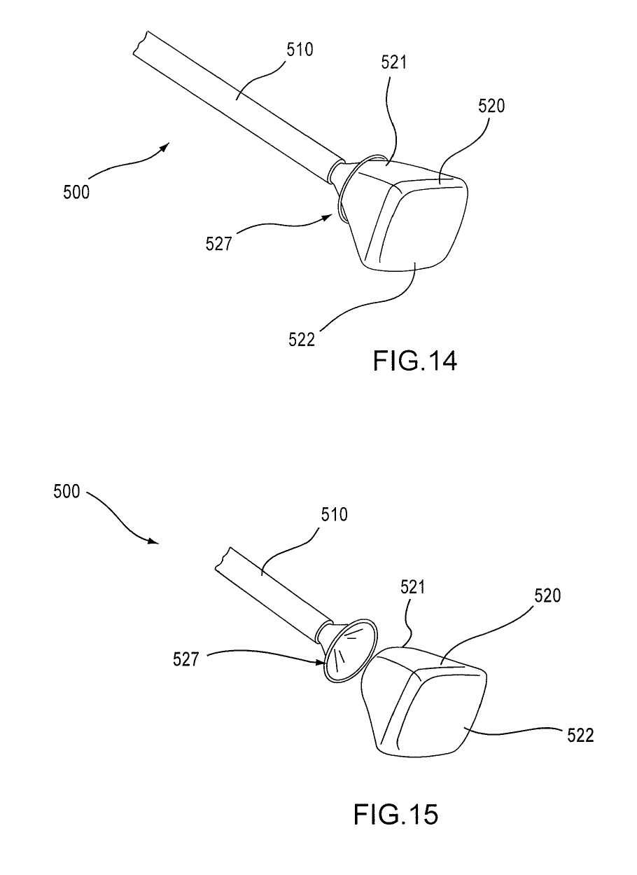

Although the expandable member 320 is shown as being detached from the shaft 310 by being severed by the distal edge 333 of the sleeve 330, in other embodiments, an expandable member can be detached from the shaft by any suitable mechanism. For example, in some embodiments, an expandable member can include a separable portion adjacent the shaft that can be actuated to detach the expandable member from the shaft. Similarly stated, although the separable portion 323 is shown as being disposed at the distal end portion of the expandable member 320, in other embodiments, the separable portion can be located within any suitable portion of the expandable member. For example, FIGS. 14 and 15 are perspective views of a portion of a medical implant device 500 according to an embodiment. The medical implant device 500 includes a shaft 510 and an expandable member 520 coupled to the shaft 510. The structure, features and/or operation of the medical implant device 500 can be similar to the structure, features and/or operation of the medical implant device 300, and are therefore not described in detail below.

The expandable member 520 has a proximal end portion 521 and a distal end portion 522. The proximal end portion 521 is coupled to the shaft 510 by any suitable means, such as a weld joint, an interference fit, an elastic coupling member, a chemical bond, or the like. The proximal end portion 521 of the expandable member 520 includes a separable portion 527. The separable portion 527 can include two edges of the side wall of the expandable member 520 that are fastened together, by a stitch, a removable staple, an adhesive or the like. In yet other embodiments, the separable portion 527 can include a dissolvable and/or degradable material that can define an opening in the side wall when actuated. Although not shown, the distal end portion 522 of the expandable member 520 can have a separable portion of the types shown and described above.

In use, the expandable member 520 can be detached from the shaft 510, as shown in FIG. 15, when the separable portion 527 is actuated. The separable portion can be actuated by any suitable mechanism, such as, for example, by a sleeve disposed at the distal end portion of the shaft 510, by a handle couple to the proximal end portion of the shaft 510 or the like. After the expandable member 520 is detached from the shaft 510, the remaining proximal portion 521 of the expandable member 520 and the shaft 510 can be extracted via the access cannula (not shown in FIGS. 14 and 15) as described above.

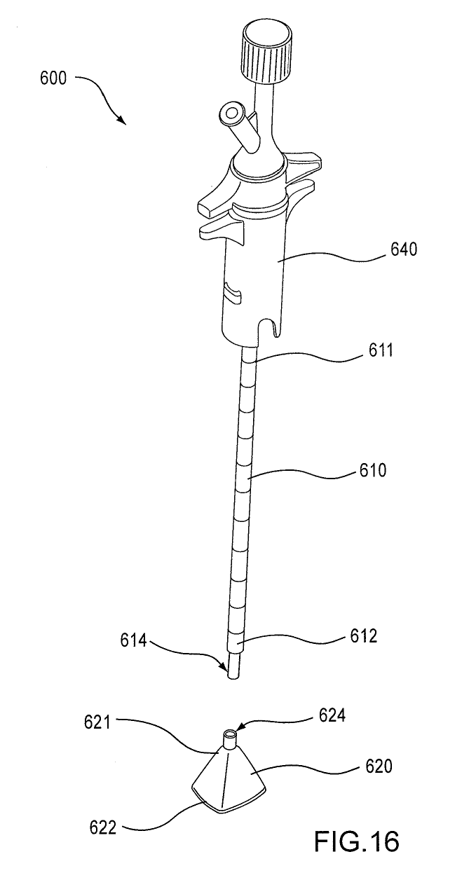

In other embodiments, an expandable member can be removably coupled to a shaft via a mechanical fastener. Similarly stated, in some embodiments, an expandable member can be rigidly coupled to the shaft. In this manner, a location, position and/or orientation of the expandable member within the body can be determined based on a location, position and/or orientation of the shaft outside of the body. For example, FIG. 16 shows a perspective view of a medical implant device 600 according to an embodiment. The medical implant device 600 includes a shaft 610 and an expandable member 620 coupled to the shaft 610. The structure, features and/or operation of the medical implant device 600 can be similar to the structure, features and/or operation of the medical implant device 300, and are therefore not described in detail below.

The shaft 610 has a proximal end portion 611 and a distal end portion 612. The distal end portion 612 includes a threaded fitting 614. Although shown as a male threaded fitting, in other embodiments, the threaded fitting 614 can be a female threaded fitting.

The expandable member 620 has a proximal end portion 621 and a distal end portion 622. The proximal end portion 621 includes a threaded fitting 624 that corresponds to and/or matingly receives the threaded fitting 614 of the shaft 610. In this manner, the expandable member 620 can be removably coupled to the shaft 610 by engagement of the threaded fitting 614 and the threaded fitting 624.

In use, the expandable member 620 can be detached from the shaft 610, as shown in FIG. 16, by rotating the distal end portion 612 of the shaft 610 relative to the expandable member 620. The distal end portion 612 of the shaft 610 can be rotated by any suitable mechanism, such as an actuator disposed within the housing 640, similar to the actuators 350 and 360 shown and described above. After the expandable member 620 is detached from the shaft 610, the shaft 610 can be extracted via the access cannula (not shown in FIG. 16) as described above.

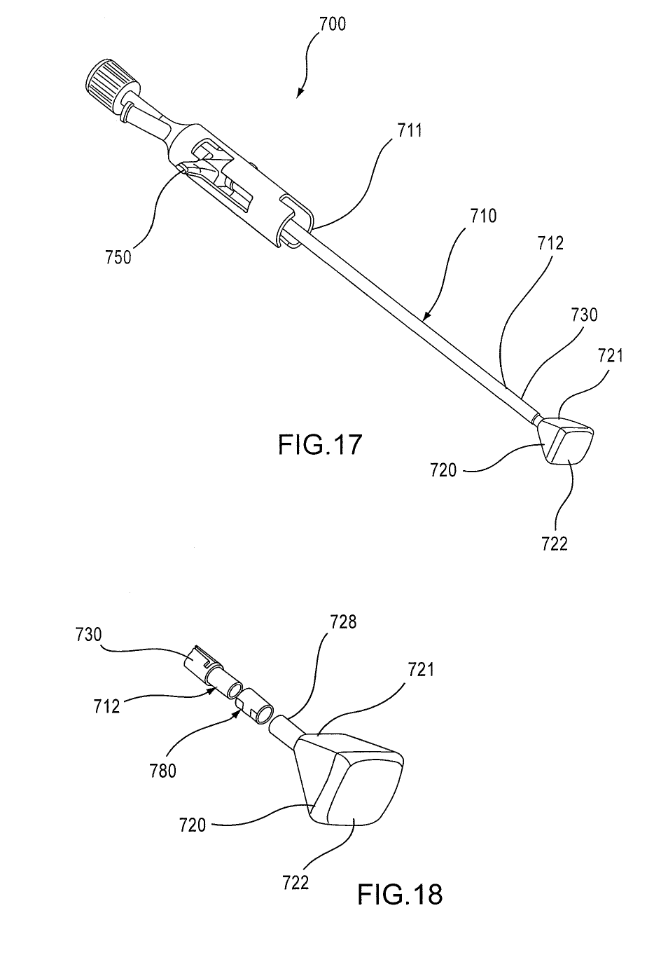

FIGS. 17-19 show another example of a medical implant device 700 according to an embodiment having an expandable member mechanically and/or removably coupled to a shaft. More particularly, the medical implant device 700 includes a shaft 710, an expandable member 720 and a compression ring 780. The structure, features and/or operation of the medical implant device 700 can be similar to the structure, features and/or operation of the medical implant device 300, and are therefore not described in detail below.

The shaft 710 has a proximal end portion 711 and a distal end portion 712. The distal end portion 712 includes a sleeve 730 that is movably coupled to the shaft 710. The sleeve 730 can be similar to the sleeve 330 shown and described above, and can be actuated from the proximal end portion 711 of the shaft 710 via the actuator 750.

The expandable member 720 has a proximal end portion 721 and a distal end portion 722. The proximal end portion 721 includes a neck 728 configured to be disposed about and/or receive the distal end portion 712 of the shaft. The compression ring 780 is disposed about the neck 728 of the expandable member 720, and is configured to couple the expandable member 720 to the distal end portion 712 of the shaft 710. Moreover, the compression ring 780 is disposed within the sleeve 730 when the sleeve is in the insertion position and the retracted position (see e.g. FIG. 17, in position to allow the expandable member to be filled). When the compression ring 780 is within the sleeve 730, the sleeve 730 maintains the compression ring 780 in a compressed configuration, thereby maintaining the coupling between the expandable member 720 and the distal end portion 712 of the shaft 710.

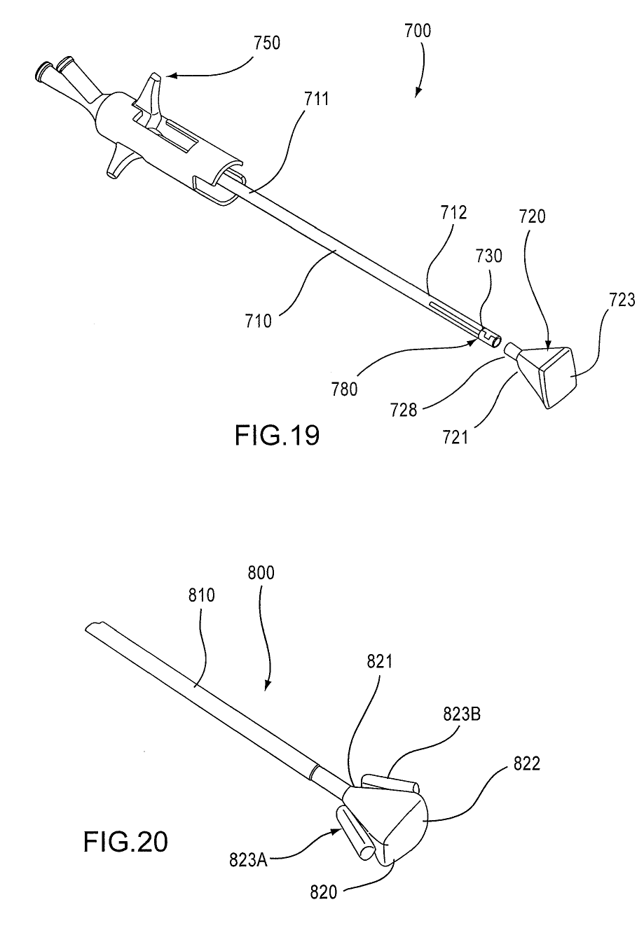

When the sleeve 730 is further retracted into a detached position (by moving the actuator 750 proximally, see e.g. FIG. 19), the sleeve 730 is disposed apart from at least a portion of the compression ring 780, thereby allowing the compression ring 780 to expand into an expanded configuration. When the compression ring 780 is in the expanded configuration, the distal end portion 712 of the shaft 710 can be removed from the neck 728 of the expandable member 720. In this manner, the expandable member 720 can be removably coupled to the shaft 710 by compression ring 780 and the sleeve 730. After the expandable member 720 is detached from the shaft 710, the shaft 710 can be extracted via the access cannula (not shown) as described above. In some embodiments, the compression ring 780 remains coupled to the sleeve 730 such that the compression ring 780 is removed from the body with the shaft 710.

Although the expandable member 320 is shown and described as including one separable portion 323 disposed at the distal end portion 322 of the expandable member 320, in other embodiments, an expandable member can include more than one separable portions. For example, FIG. 20 shows a portion of a medical implant device 800 having a shaft 810 and an expandable member 820. The expandable member 820 includes two separable portions 823A and 823B, that each extend longitudinally. Similarly stated, the separable portions 823A and 823B each extend from the distal end portion 822 of the expandable member 820 to the proximal end portion 821 of the expandable member 820. The medical implant device 800 can be configured to actuate the separable portions 823A and 823B substantially simultaneously or independently.

The expandable members shown and described herein can be any suitable expandable member. For example, in some embodiments, any of the expandable members shown and described herein can be a high-compliant medical balloon configured to significantly elastically deform when expanded and/or filled with bone cement. In other embodiments, any of the expandable members shown and described herein can be a low-compliant medical balloon configured to compact and/or displace bone material without significantly deforming. In some embodiments, for example, an expandable member can be constructed from a low-compliant material (e.g., a material having a low modulus of elasticity), such as polyamide, polyethylene terephthalate (PET), Nylons, cross-linked Polyethylene, PEBAX®, Polyurethanes, PVC or any blend of these compounds. In some embodiments, an expandable member can be constructed from Nylon 12. The compliance of a balloon is the degree to which a size of the balloon in an unfolded state changes as a function of the pressure within the balloon.

In some embodiments, any of the expandable members shown and described herein can be constructed from a material that is substantially impermeable. In other embodiments, however, any of the expandable members shown and described herein can be constructed from a porous material and/or a material that can allow a material contained therein to be conveyed from an interior region of the expandable member to a region outside of the expandable member. In this manner, such expandable members can enhance the interdigitation of the medicaments delivered into the body via the expandable member. In some embodiments, for example, an expandable member can include a flexible side wall constructed from rubber with perforated holes. In other embodiments, an expandable member can include a flexible side wall constructed from a woven fabric with meshed pores. In yet other embodiments, an expandable member can be constructed from multiple layers, with each layer being permeable to fluids. In some such embodiments, the multiple layers are laminated in such a way that the pores of the various layers do not correspond to each other. In this manner, the construction of the expandable member can be configured to control the passage of material from the interior of the expandable member to a region outside of the expandable member.

The expandable members shown and described herein can have any suitable size and/or shape. For example, in some embodiments, an expandable member can be a tube-shaped member (e.g., substantially cylindrical) when in the expanded configuration. In other embodiments, an expandable member can be shaped as a ball (e.g., substantially spherical), bag and/or sac when in the expanded configuration. In yet other embodiments, an expandable member can be shaped to correspond to an interior region of a bodily tissue (e.g., the interior region of a vertebral body) when in the expanded configuration.

Although the expandable members are shown and described herein as generally being monolithically constructed, in other embodiments, certain portions of an expandable member can be constructed separately and then be joined together to form the expandable member for use in the medical implant device. For example, in some embodiments, an expandable member can include an injection port portion that is coupled to the shaft (e.g., the shaft 310). Moreover, in some embodiments, such an injection port portion can be constructed separately and then later coupled to the main (or “holding”) portion of the expandable member.

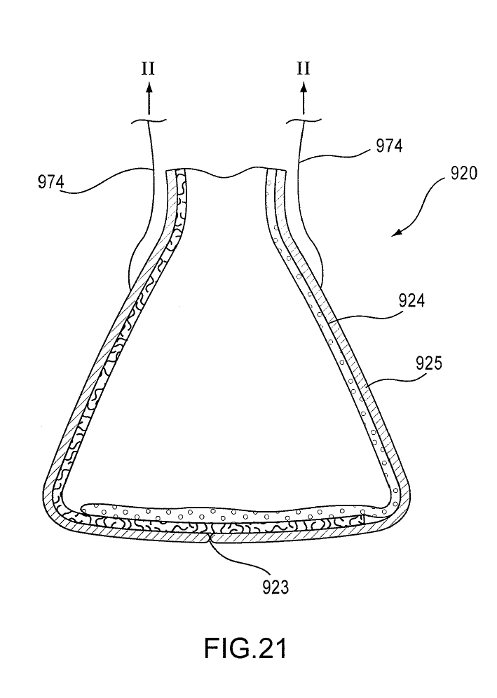

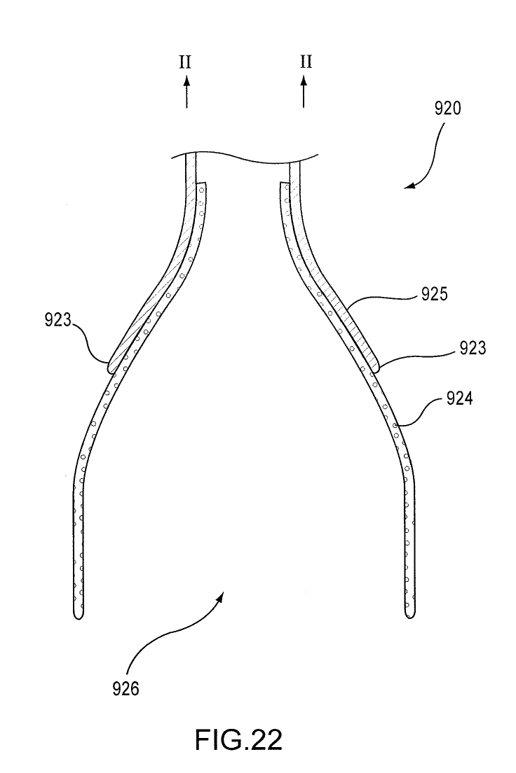

In some embodiments, an expandable member can be constructed from multiple layers. Such construction is described, for example, in U.S. Patent Publication No. 2007/0129670, entitled “Extractable Filler for Inserting Medicine into Vertebral Body,” which is incorporated herein by reference in its entirety. Another example of such an embodiment is shown in FIGS. 21 and 22, which show an expandable member 920 according to an embodiment. The expandable member 920 is constructed from a first (or inner) layer 924 and a second (or outer) layer 925. When the expandable member 920 is in a first expanded configuration, a first portion of the inner layer 924 forms an injection port portion that is coupled to a shaft, tube or other mechanism for conveying a medicament into the expandable member 920. A second (or distal end) portion of the inner layer 924 is folded, thereby forming a double-layer construction.

The outer layer 925 has a separable portion 923 that defines a selectively releasable opening 926. The separable portion 923 can include any mechanism for being joined, coupled and/or for forming the selectively releasable opening 926, as described herein. The separable portion 923 of the outer layer 925 is actuated via two filaments or threads 974. In particular, although the threads 974 are configured to actuate the separable portion 923, they are coupled to the expandable member 920 at a location spaced apart from the separable portion 923. In this manner, as described below, at least a portion of the medicament within the expandable member can be released from the expandable member by exerting a force on the proximal (or “free”) end of the expandable member.

In use, to move the expandable member 920 from the first expanded configuration (FIG. 21) to the second expanded configuration (FIG. 22), the threads 974 are moved proximally, as shown by the arrows II. The force exerted by the threads 974 on the outer layer 925 cause the opening 926 to be selectively released, opened and/or defined. Additionally, the outer layer 925 moves proximally relative to the inner layer 924, further expanding the opening 926 (as shown in FIG. 22). When the outer layer 925 is moved apart from the distal end portion of the expandable member 920, the folded portion of the inner layer 924 can unfold (under the influence of the internal pressure within the expandable member), thereby allowing at least portion of the medicament contained therein to be conveyed outside of the expandable member.

In some embodiments, an expandable member can include a separable portion defining an opening configured to have a variable size. In this manner, the flow rate and/or amount of medicament conveyed from the expandable member can be controlled by the actuation mechanism. In some embodiments, for example, a separable portion can define an opening that is stitched and/or fastened closed by several discrete stitches or fasteners. In such an embodiment, the size of the opening produced and/or released when the separable portion is actuated can depend on the number of stitches and/or fasteners removed.

Any of the medical implant devices described herein can be used to deliver a medicament, a pasty medicament, a therapeutic substance and/or a structural member into the body of an animal. For example, in some embodiments, a method includes inserting an expandable member into a bodily tissue of a body, such as, for example, a vertebral body. The expandable member, which can be any expandable member of the type shown and described herein, defines a selectively releasable opening. A medicament is conveyed into the expandable member via a shaft coupled to the expandable member to at least partially fill the expandable member. The medicament is then allowed to at least partially solidify within the expandable member. The method then includes detaching and/or severing the expandable member from the shaft. The shaft can then be removed from the body, thereby leaving the medicament (e.g., the structural material) and the expandable member within the bodily tissue.

In some embodiments, the method can optionally include moving the expandable member within the bodily tissue after the conveying. This can be accomplished, for example, by moving the shaft. Such movement can allow the expandable member and the medicament therein to be placed in a desired location, position and/or orientation within the bodily tissue.

In some embodiments, the method can optionally include selectively releasing the opening defined by the expandable member. The opening can be released by any of the mechanisms described herein. In this manner, a portion of the medicament can be conveyed from the expandable member into direct contact with a portion of the bodily tissue. Such contact can enhance the efficacy of the medicament, the interdigitation of the bone cement within the bodily tissue or the like.

In some embodiments, the method can include defining a void within the bodily tissue before the medicament is conveyed into the expandable member. The void can be defined by any suitable mechanism or tool. For example, in some embodiments, the void can be defined by a separate tool configured to define a void having the desired size and/or shape (e.g., a size or shape that corresponds to the size or shape of the expandable member in its expanded configuration). In other embodiments, the void can be defined by the medical implant device, for example, by moving the sheath and/or shaft of the device distally. In this manner, the void can receive a portion of the medicament that is conveyed from within the expandable member. In some embodiments, the void can have a length that is greater than a length of the expandable member.

Although the method described above includes conveying the medicament into the expandable member in one substantially continuous operation, in other embodiments, the medicament can be conveyed into the expandable member in multiple discontinuous operations (e.g., at different times). For example, in some embodiments, a method includes inserting an expandable member into a bodily tissue of a body, such as, for example, a vertebral body. A separable portion of the expandable member is actuated to selectively release an opening defined therein. A first amount of a medicament is conveyed into the expandable member via a shaft coupled to the expandable member to at least partially fill the expandable member, and to allow a portion of the medicament to be conveyed outside of the expandable member and into contact with the bodily tissue. The method then includes moving the expandable member proximally (i.e., withdrawing) by a first distance. A second amount of the medicament (or of a different medicament) is conveyed into the expandable member via the shaft. The expandable member is moved proximally by a second distance, and a third amount of the medicament (or of a different medicament) is conveyed into the expandable member via the shaft. In some embodiments, the method includes severing the expandable member from the shaft.

Methods that include multiple fill operation and/or moving the expandable member within the bodily tissue can allow an amount of medicament greater than the volume of the expandable member (when in the expanded configuration) to be conveyed into the bodily tissue.

In some embodiments, a method includes inserting into a vertebra of a body an expandable member. The expandable member defines a selectively releasable opening, which can be, for example, disposed at a distal end portion of the expandable member. A first volume of a medicament is conveyed into the expandable member via a shaft coupled to the expandable member to at least partially fill the expandable member. The expandable member is moved proximally by a first distance within the vertebra. A second volume of the medicament is conveyed into the expandable member via the shaft. In some embodiments, the second volume of medicament can include a different medicament (e.g., a medicament configured to solidify faster than the first medicament conveyed into the expandable member). The expandable member is moved proximally by a second distance within the vertebra. The second distance can be different than the first distance. The expandable member is detached from the shaft, and the shaft is removed from the body.

Although the method described above includes detaching the expandable member from the shaft, in other embodiments, a method includes removing the expandable member from the body. For example, in some embodiments, a method includes inserting into a vertebra an expandable member. A first volume of a first medicament is conveyed into the expandable member via a shaft coupled to the expandable member to at least partially fill the expandable member. A separable portion of the expandable member is actuated to selectively release an opening defined therein. The expandable member is then moved proximally a first distance within the vertebra. In this manner, a portion of the first volume of the first medicament can be conveyed from the expandable member. A second volume of a second medicament is conveyed into the expandable member via the shaft. The expandable member is then removed from the vertebra.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, although the separable portion 323 of the expandable member 320 is shown as being actuated by the cap 373, in other embodiments, the separable portion 323 of the expandable member 320 can be actuated by any suitable mechanism. In some embodiments, for example, the separable portion 323 of the expandable member 320 can be actuated by the sleeve 330, the bone cement injector 302 or the like.

Although the separable portions of the expandable members are shown and described above as being mechanically actuated, in other embodiments, a separable portion can be actuated by any suitable mechanism. For example, in some embodiments, a device can include a cauterizing cutter configured to remove an expandable member from a shaft. In such embodiments, a dielectric coating can be applied to the inside of the sleeve and can be electrically charged using the shaft as a conduit (e.g., a bipolar electrical connection).

In some embodiments, an expandable member can be coupled to a shaft via a connection that is dissolvable when exposed to the medicament (e.g., bone cement) conveyed into the expandable member. In yet other embodiments, the entire expandable member is dissolvable. In some embodiments, a portion or all of the expandable member can be constructed from PLA, PGA, Alpha and Beta TCP or the like. In yet other embodiments, all or a portion of the expandable member can be bioresorbable.

In some embodiments, an expandable member can be coupled to a shaft via a nitinol collar. In use, the collar can be changed from an “attached” configuration to a “detached” configuration by heat generated by an exothermic reaction resulting from the curing of the bone cement.

In some embodiments, an expandable member can be coupled to a shaft via a “J” slot style attachment.

In some embodiments, an expandable member can be coupled to a shaft via a pressure sensitive connection. In such embodiments, the connection between the expandable member and the shaft connection is maintained while the expandable member and/or the connection are under pressure, but is released when pressure is relieved from the expandable member and/or the connection

In some embodiments, an expandable member can be coupled to a shaft by a directional connection. For example, in some embodiments a connector can maintain the coupling between the expandable member and the shaft when the connector is in compression, and the connector can release the coupling between the expandable member and the shaft when the connector is in tension. In such embodiments, the connector can be actuated by a pull cord, tether, filament or rod that extends to the proximal end portion of the shaft.

In some embodiments, an expandable member can be coupled to a shaft by a deformable connector. Such a connector may include deformable protrusion or teeth (e.g., similar to the protrusions used to removably couple a medical bandage to a limb). The teeth can be deformed to release the connection.

In some embodiments, an expandable member can be coupled to a shaft by an adhesive or glue bond that is weak in shear/tension.

In some embodiments, an expandable member can include an embedded “cheese wire” in the neck thereof. In such embodiments, actuation of the “cheese wire” can separate, sever and/or open the portion of the expandable member adjacent the shaft, thereby releasing the expandable member from the shaft.

Although various embodiments have been described as having particular features and/or combinations of components, other embodiments are possible having a combination of any features and/or components from any of embodiments where appropriate. For example, in some embodiments, a medical implant device can include a detachment mechanism similar to the detachment mechanism 180 and an expandable member with a separable portion, similar to the expandable member 320.

1. An apparatus, comprising:
a shaft having a proximal end portion and a distal end portion;
an expandable member configured to move between a collapsed configuration and an expanded configuration, the expandable member being coupled to the distal end portion of the shaft and being configured to receive a medicament therein;
a sleeve coupled to the distal end portion of the shaft and configured to move relative to the shaft between a first position and a second position, the sleeve being configured to be disposed about the expandable member when the sleeve is in the first position and disposed apart from the expandable member when the sleeve is in the second position; and
a detachment mechanism coupled to the distal end portion of the shaft, the detachment mechanism configured to detach the expandable member from the shaft when the sleeve is in the second position.

2. The apparatus of claim 1, wherein a portion of the expandable member defines a selectively releasable opening, the apparatus further comprising:
a thread coupled to the expandable member, the thread configured to releasably fasten the opening such that the portion of the expandable member is substantially leakproof when the expandable member is in a first expanded configuration and the medicament can pass through the opening when the expandable member is in a second expanded configuration.

3. The apparatus of claim 1, wherein the expandable member is configured to selectively convey a portion of the medicament.

4. The apparatus of claim 3, wherein the medicament is at least one of a liquid medicament, a pasty medicament, a therapeutic substance or a structural member.

5. The apparatus of claim 1, further comprising:
a retention member coupled between the expandable member and the shaft, the retention member configured to sever when the sleeve is moved from the first position to the second position.

6. The apparatus of claim 1, wherein the medicament is a mixture of a liquid and at least one of a medicinal powdered substance or a medicinal granular substance.

7. The apparatus of claim 1, further comprising:
an injection tool configured to inject the medicament into the expandable member through the shaft.

8. The apparatus of claim 1, wherein the expandable member includes a flexible and hermetic wall, the flexible and hermetic wall defining a first portion coupled to the shaft, and a second portion defining a selectively releasable opening.

9. The apparatus of claim 1, wherein the expandable member is configured to be inserted into a vertebra during a surgical procedure when the expandable member is in the collapsed configuration, the expandable member configured to remain in the vertebra when the expandable member is in the expanded configuration, a portion of the medicament configured to contact the vertebra when the expandable member is in the expanded configuration.

10. An apparatus, comprising:
a shaft having a proximal end portion and a distal end portion;
an expandable member configured to move between a collapsed configuration and an expanded configuration, the expandable member being coupled to the distal end portion of the shaft; and
a sleeve coupled to the distal end portion of the shaft and configured to move relative to the shaft between a first position, a second position and a third position, the sleeve being configured to be disposed about the expandable member when the sleeve is in the first position and disposed apart from the expandable member when the sleeve is in the second position, the sleeve configured to sever a portion of the expandable member when the sleeve is in the third position.

11. The apparatus of claim 10, wherein a portion of the expandable member defines a selectively releasable opening when the expandable member is in the expanded configuration, the apparatus further comprising:
a thread coupled to the expandable member, the thread configured to releasably fasten the opening such that the portion of the expandable member is substantially leakproof when the expandable member is in a first expanded configuration and a medicament contained within the expandable member can pass through the opening when the expandable member is in a second expanded configuration.

12. The apparatus of claim 10, wherein the expandable member is configured to receive a medicament therein.

13. The apparatus of claim 12, wherein the medicament is at least one of a liquid medicament, a pasty medicament, a therapeutic substance or a structural member.

14. The apparatus of claim 10, further comprising an injection tool configured to inject the medicament into the expandable member through the shaft.

15. The apparatus of claim 10, wherein the expandable member includes a flexible and hermetic wall, the flexible and hermetic wall defining a first portion coupled to the shaft, and a second portion defining a selectively releasable opening.

16. A method, comprising:
inserting into a bodily tissue of a body an expandable member defining a selectively releasable opening;
conveying a medicament into the expandable member via a shaft coupled to the expandable member to at least partially fill the expandable member;
allowing the medicament to at least partially solidify within the expandable member;
detaching the expandable member from the shaft; and
removing the shaft from the body.

17. The method of claim 16, further comprising moving the expandable member within the body after the conveying.

18. The method of claim 16, wherein when the conveying includes conveying one of a liquid medicament, a pasty medicament, a therapeutic substance or a structural member.

19. The method of claim 16, further comprising:
releasing the selectively releasable opening such that a portion of the medicament directly contacts the bodily tissue.

20. The method of claim 16, further comprising:
before the conveying the medicament, defining a void distal to the expandable member.

21. The method of claim 16, further comprising;
before the inserting, defining a void within the bodily tissue, the void having a length greater than a length of the expandable member.

22. A method, comprising:
inserting into a vertebra of a body an expandable member defining a selectively releasable opening;
conveying a first volume of a medicament into the expandable member via a shaft coupled to the expandable member to at least partially fill the expandable member;
actuating the selectively releasable opening such that a portion of the medicament is conveyed from the expandable member into the vertebra;
conveying a second volume of the medicament into the expandable member via the shaft;
detaching the expandable member from the shaft; and
removing the shaft from the body.

23. The method of claim 22, wherein the conveying the second volume includes conveying the second volume such that at least a portion of the first volume of the medicament is moved into direct contact with the vertebra.

24. The method of claim 22, wherein the first volume is different from the second volume.

25. The method of claim 22, further comprising:
moving the expandable member proximally a first distance within the vertebra after the actuating; and
moving the expandable member proximally a second distance within the vertebra after the conveying the second volume, the first distance different than the second distance.

26. A method, comprising:
inserting into a vertebra an expandable member defining a selectively releasable opening;
conveying a first volume of a first medicament into the expandable member via a shaft coupled to the expandable member to at least partially fill the expandable member;
actuating the selectively releasable opening;
conveying a second volume of a second medicament into the expandable member via the shaft such that a portion of the medicament is conveyed from the expandable member into the vertebra; and
removing the expandable member from the vertebra.

27. The method of claim 26, wherein the selectively releasable opening is disposed at a distal end portion of the expandable member, the actuating includes actuating the selectively releasable opening at a first time such that the selectively releasable opening is characterized by a first size, the method further comprising:
actuating the selectively releasable opening at a second time before the removing such that the selectively releasable opening is characterized by a second size greater than the first size.

28. The method of claim 26, further comprising:
moving the expandable member proximally a first distance within the vertebra after the actuating.

29. The method of claim 26, wherein the shaft is rigidly coupled to the expandable member such that a position of the expandable member is fixed relative to a position of the shaft.

30. A method, comprising:
inserting into a vertebra of a body an expandable member defining a separable portion;
conveying a first volume of a medicament into the expandable member via a shaft coupled to the expandable member to at least partially fill the expandable member;
actuating the separable portion to define an opening;
conveying a second volume of the medicament into the expandable member via the shaft such that a first portion of the medicament is conveyed from the expandable member into contact with a bodily tissue within the vertebra;
moving the expandable member proximally a first distance within the vertebra;
conveying a third volume of the medicament into the expandable member via the shaft such that a second portion of the medicament is conveyed from the expandable member into contact with the bodily tissue within the vertebra;
moving the expandable member proximally a second distance within the vertebra;
conveying a fourth volume of the medicament into the expandable member via the shaft such that a third portion of the medicament is conveyed from the expandable member into contact with the bodily tissue within the vertebra;
detaching the expandable member from the shaft; and
removing the shaft from the body.

31. The method of claim 30, wherein the actuating includes actuating at a first time to define the opening having a first size, the method further comprising:
actuating the separable portion at a second time to define the opening having a second size greater than the first size.

32. The method of claim 30, wherein the first volume is different from the second volume, the third volume and the fourth volume.

33. The method of claim 30, wherein the first distance is different from the second distance.

34. A method, comprising:
inserting into a vertebra an expandable member defining a selectively releasable opening;
conveying a first volume of a first medicament into the expandable member via a shaft coupled to the expandable member to at least partially fill the expandable member;
actuating the selectively releasable opening;
moving the expandable member proximally a first distance within the vertebra;
conveying a second volume of a second medicament into the expandable member via the shaft; and
removing the expandable member from the vertebra.

35. A method, comprising:
inserting into a vertebra an expandable member defining a selectively releasable opening;
conveying a first volume of a first medicament into the expandable member via a shaft coupled to the expandable member to at least partially fill the expandable member;
actuating the selectively releasable opening;
conveying a second volume of a first medicament via the shaft coupled to the expandable member such that a portion of the medicament is conveyed from the expandable member into contact with a bodily tissue within the vertebra;
moving the expandable member proximally a first distance within the vertebra;
conveying a third volume of a second medicament into the expandable member via the shaft; and
removing the expandable member from the vertebra.
